(19) United States
(12) Patent Application Publication
(10) Pub. No.: US 2010/0233718 A1
(43) Pub. Date: Sep. 16, 2010

(54) NON-INVASIVE TECHNIQUE FOR CONDUCTING SKIN INFLAMMATORY DISEASE PHARMACO-GENOMIC STUDIES AND DIAGNOSES THEREOF

(75) Inventors: Jérôme AUBERT, Grasse (FR); Johannes Voegel, Chateauneuf/Grasse (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, Biot (FR)

(21) Appl. No.: 12/765,284

(22) Filed: Apr. 22, 2010

Related U.S. Application Data
(63) Continuation of application No. PCT/EP2008/064551, filed on Oct. 27, 2008.

(51) Int. Cl. C12Q 1/68; C07K 14/54; C07K 2/00; C07K 14/00; C07K 14/545

(52) U.S. Cl. 435/6; 530/351; 530/300; 530/350

(57) ABSTRACT

Non-invasive techniques to conduct skin inflammatory disease pharmaco-genomic studies and diagnoses thereof feature discriminating biomarkers and genes and in vitro diagnostic methods employing such biomarkers.

### CROSS-REFERENCE TO ALL PRIOR APPLICATIONS

This application claims priority under 35 U.S.C. §120 of U.S. Provisional Application No. 60/996,073, filed Oct. 26, 2007, and is a continuation of PCT/EP 2008/064551, filed Oct. 27, 2008 and designating the United States (published in the English language on Apr. 30, 2009 as WO 2009/053493 A1), each hereby expressly incorporated by reference in its entirety and each assigned to the assignee hereof.

### BACKGROUND OF THE INVENTION

1. Technical Field of the Invention

The present invention relates to non-invasive techniques for conducting skin inflammatory disease pharmaco-genomic studies and diagnoses thereof and, particularly, scalp psoriasis. This Invention also features discriminating biomarkers and genes and in vitro diagnostic methods employing such biomarkers.

2. Description of Background and/or Related and/or Prior Art

The diagnosis and management of patients with inflammatory skin disease/disorders remains a very challenging and rewarding aspect of ‘core’ dermatology practice. Inflammatory skin diseases/ disorders affect men, women and children of all races. They consist of a broad category ranging in severity and etiopathogeny. They include very common dermatoses such as Psoriasis, Eczema, Atopic dermatitis, Acne, Rosacea, but also less frequent or rare diseases such as lichenoid eruptions or erythrodermia. While these diseases/disorders are usually not generally perceived to be serious or life threatening, they can significantly impact on the quality of life for sufferers. Appropriate treatment is based on correct diagnosis.

As described in the following text, one specific embodiment of the present invention is diagnosis of psoriasis and particularly scalp psoriasis.

Psoriasis is a common chronic skin disorder estimated to affect about 2% of the Western population, with the scalp being the most common site of involvement at the onset and throughout the course of the disease (Van de Kerkhof P C, Franssen M E. Psoriasis of the scalp. Diagnosis and management. Am J Clin Dermatol 2001; 2: 159-165; Farber E M, Nall L. Natural history and treatment of scalp psoriasis. Cutis 1992; 49: 396-400.). Indeed, 50% to 80% of patients with psoriasis report scalp psoriasis or concomitant psoriasis of the scalp and the body, leading to a prevalence of scalp psoriasis of 1.5% to 2% in northwestern Europe. For many patients, psoriasis of the scalp is the most difficult aspect of their disease owing to the visibility of lesions.

Psoriasis is a chronic, inflammatory skin disorder, which is thought to have an immune-mediated pathogenesis whereby activated T cells infiltrate the dermis and stimulate cytokines, thus promoting keratinocyte proliferation (Krueger G. The immunologic basis for the treatment of psoriasis with new biologic agents. J Am Acad Dermatol 2002; 46: 1-23.). Scalp psoriasis does not generally result in haft loss, although some increased shedding of telogen hairs and reduction in hair density is common in psoriasis plaques.

In addition, extensive hair loss can occur in the erythrodermic forms of psoriasis, and chronic severe hyperkeratotic scalp psoriasis may induce scarring alopecia (Bardazzi F, Fanti P A, Orlandi C, Chieregato C, Misciali C. Psoriatic scarring alopecia: observations in four patients. Int J Dermatol. 1999 October; 38(10):765-8.). In addition, the morphology of hair follicles was examined in psoriatic scalp biopsies and compared with normal scalp. In scalp psoriasis the lower outer root sheath and hair matrix were not affected by the psoriatic changes, although there was an irregular expansion in the proximal lower outer root sheath. This area has been characterized, by the presence of keratin K19-containing cells, as the putative stem cell region. (Wilson C L et al.; 4: Br J Dermatol. 1994 August; 131(2):191-200 “Keratinocyte differentiation in psoriatic scalp: morphology and expression of epithelial keratins”).

Another study analyzed anagen hair follicles obtained from both healthy (n=7) and uninvolved psoriatic (n=4) scalps were segmentally analyzed for proliferative activity using DNA flow cytometry. Hair follicle kinetics were almost equal in either group except for the infundibular portion which exhibited significant increase of S-phase values in psoriatic patients. Maximum proliferation was disclosed within the bulbar segment. This study confirms that cell kinetics behavior of hair follicles from uninvolved scalp of psoriatics compared with those from healthy scalps is altered in the infundibular portion only. (Katsuoka K, et al. 6: Dermatologica. 1987; 174(3):105-9. “Cell kinetics of the human anagen hair follicle. Flow cytometric studies in healthy and psoriatic subjects”).

A definitive diagnosis of scalp psoriasis may be difficult in cases where there is a clinical overlap with seborrhoeic dermatitis. Nevertheless, microscopic examination of the scalp can be used to confirm a diagnosis of scalp psoriasis, because a characteristic histological appearance associated with the condition has been observed, represented by proliferation of parakeratotic cells, sometimes accompanied by leucocyte infiltration (Conti Diaz I A, Civila E, Veiga R. The importance of microscopic examination in the management of esquamative diseases of the scalp. Mycopathologia 2002; 153: 71-75.). Persistent scaly plaques on a bald scalp occasionally require histological examination to exclude Bowen's disease. An underlying mycotic infection or allergic contact dermatitis (although the latter is less frequent) should be ruled out prior to a diagnosis of scalp psoriasis being made (Elewski B E. Clinical diagnosis of common scalp disorders. J Invest Dermatol Symp Proc 2005; 10: 190-193.; Larko O. Problem sites: scalp, palm and sole, and nail. Dermatol Clin 1995; 13: 771-777.).

According to this prior art no pharmaco-genomics investigations have studied skin inflammatory lesions from hair samples, collecting by non-invasive method, including the scalp psoriatic lesions.

### SUMMARY OF THE INVENTION

The present invention provides a non-invasive method to perform skin inflammatory disease pharmaco-genomic studies and particularly scalp psoriasis and a diagnostic method thereof. This invention also features discriminating biomarkers and gene and in vitro diagnostic techniques employing such biomarkers.

Thus, a method has now been developed to perform non-invasive pharmaco-genomic studies which is applicable to affected skin including the scalp for any type of disease, and for any type of pharmacological agent (small molecule drugs; biologics) and any type of application (topical and systemic) as well.

Therefore, in one embodiment, the present invention features a method to perform non-invasive pharmaco-genomic studies of affected skin and/or scalp comprising collecting hair follicles non-invasively and analyzing gene expression profiling therein.

Within this study, changes in gene transcription in hair follicles were investigated (collected by a non-invasive method).

By “hair” is meant all kinds of hair present on the body skin, including scalp hair.

By “non-invasive” method is meant any method which does not require surgical procedures.

In a specific embodiment, the present invention features a method to perform non-invasive pharmaco-genomic studies of affected scalp comprising collecting hair follicles non-invasively and analyzing gene expression profiling. Particularly, the method can be used to assess at least one of these parameters:

i) identify genes allowing to discriminate affected samples to healthy volunteers samples,

ii) identify early markers monitoring a compound or drug efficacy,

iii) characterize compound or drug anti-inflammatory mechanism,

iv) clusterize responders versus non-responders based on large scale gene expression profiling.

### DETAILED DESCRIPTION OF BEST MODE AND SPECIFIC/PREFERRED EMBODIMENTS OF THE INVENTION

In a particular embodiment of the invention, a study was conducted to investigate changes in gene transcription in hair follicles (collected by a non-invasive method) of the psoriatic scalps being treated with clobex 0.05%, versus healthy volunteers.

For instance, the hair follicles are plucked with tweezers from the scalp. The gene expression profiling (real time PCR and large scale gene expression array) is used in an effort to i) identify genes allowing to discriminate affected samples to healthy volunteers samples, ii) identify early markers monitoring Clobex 0.05% efficacy, iii) characterize its anti-inflammatory mechanism, iv) clusterize responders versus non-responders based on large scale gene expression profiling.

In another embodiment, the present invention concerns a non-invasive diagnosis method of inflammatory skin disease or disorders and in a specific embodiment diagnoses of psoriasis on scalp comprising the steps of:

collect non-invasively hair follicles,

study/determine the gene expression by analysis method,

clusterize control samples to skin affected patient(s') samples (specific embodiment psoriatic samples) based discriminating genes.

It is understood by “control” samples, are intended samples (hairs samples) collected from subject(s) in healthy conditions or in non-involved inflammatory skin conditions.

In a preferred embodiment, the said non-invasive diagnostic method of inflammatory skin disease or disorders comprises discriminating genes/ markers (including proteins) which are selected from the well known inflammatory specific genes and/or markers. Particularly, the discriminating genes/markers are selected from the following:

Keratin 16 (KRT16); gap junction protein, beta 2, (connexin 26) (GJB2); chitinase 3-like 2 (CHI3L2); interleukin 8 (IL8); fatty acid binding protein 5 (FABP5); interleukin 1, beta (ID1B); signal transducer and activator of transcription (STAT1); heparanase (HPSE); solute carrier family 6 (amino acid transporter), member 14 (SLC6A14); transcobalamin I (vitamin B12 binding protein, R binder family) (TCN1); tumor necrosis factor (TNF); interleukin 1 family, member 5 (delta) (IL1F5); small proline-rich protein 2D (SPRR2D); kallikrein 13 (KLK13); chemokine (C-X-C motif) ligand 10 (CXCL10); desmoglein 3 (pemphigus vulgaris antigen) (DSG3); S100 calcium binding protein A12 (S100A12); interleukin 1 receptor antagonist (IL1RN); superoxide dismutase 2, mitochondrial (SOD2); keratin 6C; (KRT6E); interferon-induced protein with tetratricopeptide repeats 3 (IFIT3); desmocollin 2 (DSC2); endothelial cell growth factor 1 (platelet-derived) (ECGF1); RAS guanyl releasing protein 2 (calcium and DAG-regulated) (RASGRP2); wingless-type MMTV integration site family, member 5A (WNT5A); myxovirus (influenza virus) resistance 1, interferon-inducible protein p78 (mouse) (MX1); small proline-rich protein 1A (SPRR1A); defensin, beta 4 (DEFB4); S100 calcium binding protein A9 (S100A9); interleukin 1 family, member 9 (IL1F9); kallikrein 6 (neurosin, zyme) (KLK6); matrix metallopeptidase 9 (MMP9); serpin peptidase inhibitor, clade B (ovalbumin), member 3 (SERPINB3); interferon, gamma (IFNG); lipocalin 2 (oncogene 24p3) (LCN2); interferon, alpha-inducible protein 27 (IFI27); peroxisome proliferator-activated receptor delta (PPARD); serpin peptidase inhibitor, clade B (ovalbumin), member 1 (SERPINB1); latent transforming growth factor beta binding protein 1 (LTBP1); pre-B-cell colony enhancing factor 1 (PBEF1); transglutaminase 1 (K polypeptide epidermal type I, protein-glutamine-gamma-glutamyltransferase) (TGM1); chemokine (C-C motif) ligand 20 (CCL20); aldo-keto reductase family 1, member B10 (aldose reductase) (AKR1B10); S100 calcium binding protein A7 (S100A7).

In a specific embodiment, the said non-invasive diagnose psoriasis method comprises discriminating genes which are selected from the following: interleukin 8 (IL8); beta 4defensin (DEFB4); S100 calcium binding protein A7 (S100A7); S100 calcium binding protein A9 (calgranulin B) (S100A9); S100 calcium binding protein A12 (S100A12); interleukin 1b (IL-1b); lipocalin 2 (oncogene 24p3) (LCN2); transcobalamin I (vitamin B12 binding protein, R binder family) (TCN1); Interferon alpha-inducible protein 27 (IFI27); Peroxisome proliferator-activated receptor- [symbol] PPAR-δ); serpin peptidase inhibitor, clade B (ovalbumin), member 3 (SERPIN B3).

In the context of the present invention <<gene>> refers to nucleic acid or nucleotide sequence encoding for a protein/biomarker expression, and the proteins related to the said gene.

In addition, the present invention features the said gene expression product as “biological targets”. By “target” is understood an enzyme, a receptor, other protein or mRNA that can be modified by an external stimulus. The definition is context-dependent and can refer to the biological target of a pharmacologically active drug compound, or the receptor target of a hormone. The implication is that a molecule is “hit” by a signal/stimulus and its behavior is thereby changed.

In the context of invention, target of interest are those above mentioned expression products.

By “analyzing method” is meant any method or technique carried out to determine gene expression levels. Those are generally well known by one skilled in the art and are determined according to transcription or translation rates. By transcription rate is understood, mRNA levels. By translation is meant, protein production rate.

Gene expression products/Biomarkers (e.g., Proteins) might be determined by any appropriate methods such as western-blot, IHC, MAS spectrometry analysis (MAldi-TOF and LC/MS analysis), Radioimmunoassay (RIA), Elisa or by any other methods well known by those skilled in the art or by mRNA dosage by any appropriate methods well known to those skilled in the art.

For instance, it can be mentioned quantitative or semi-quantitative methods for mRNA of gene of interest detection are well known by one skilled in the art.

Methods based on mRNA hybridation with nucleic probes are typically known (Northern Blot, RT-PCR, RNase protection). It might be advantageous to use detection markers such as fluorescent, radio-labeled, enzymatic agents or other ligands (for example avidine/biotine).

Gene translation rate may also be assessed by immunological assays of gene expression product. Accordingly, polyclonal or monoclonal antibodies may be employed. Antibodies manufacturing methods are well known to one skilled in the art. For instance, monoclonal antibody might be produced according to Kohler and Milstein method (Nature (London), 256: 495-497 (1975) or by cloning a nucleic acid expression clone in hybridoma.

Immunological dosages are assessed by solid or homogeny phase, in one or two time frames; with the so-called sandwich method or with competition method.

In a preferred embodiment, the determination technique is Real time PCR.

Another embodiment of the invention features monitoring efficacy of a pharmacological agent in preventing or treating inflammatory skin disease/disorders (in a specific embodiment scalp psoriasis) comprising the steps of:

administer to a patient in need of treatment a therapeutically effective amount of a pharmacological agent,

collect non-invasively hair follicles,

study/determine the gene expression by analyzing method,

analyze skin affected patient(s') samples to controls' samples or to previous skin affected patient(s') samples without pharmacological agent

The pharmacological agent is selected from a small molecule drug or a biological agent.

The present invention also features a method to monitor skin (or scalp in a specific embodiment) inflammation in a skin affected patient(s) (in a specific embodiment psoriatic patient) comprising the steps of:

collect non-invasively patient's hair follicles,

study/determine the gene expression by analyzing method,

analyze inflammation gene (s) from said inflammatory skin affected patient(s') samples (specific embodiment patient's psoriatic samples) to controls' samples or to previous patient psoriatic samples.

Another embodiment of the invention features a predictive model of inflammatory skin affected patient(s') (in a specific embodiment psoriatic scalp) determination comprising monitoring the modulation in expression of selected discriminating biomarkers/genes.

By “modulation in expression” is meant a change in the expression of selected genes and/or said biomarkers/gene expression products levels and/or their activities in comparison with healthy volunteers and encompasses either a down regulation/under expression or up regulation/over expression.

By “analyzing method” is meant any method carried out to determine gene expression levels. Those are generally well known to one skilled in the art and are determined according to transcription or translation rates. By transcription rate is understood, mRNA levels. By translation is meant, protein production rate.

Gene expression products/Biomarkers (e.g., Proteins) might be determined by any appropriate methods such as western-blot, IHC, MAS spectrometry analysis (MAldi-TOF and LC/MS analysis), Radioimmunoassay (RIA), Elisa or by any other methods well known by skilled in the art or by mRNA dosage by any appropriate methods well known by skilled in the art.

In a preferred embodiment, modulation of at least 1 discriminating genes or markers selected from the inflammatory markers are monitored. The discriminating genes or markers are preferentially selected from the following: interleukin 8 (IL8); beta 4defensin (DEFB4); S100 calcium binding protein A7 (S100A7); S100 calcium binding protein A9 (calgranulin B) (S100A9); S100 calcium binding protein A12 (S100A12); interleukin 1b (IL-1b); lipocalin 2 (oncogene 24p3) (LCN2); transcobalamin I (vitamin B12 binding protein, R binder family) (TCN1); Interferon alpha-inducible protein 27 (IFI27); Peroxisome proliferator-activated receptor- [symbol] (PPAR-δ); serpin peptidase inhibitor, clade B (ovalbumin), member 3 (SERPIN B3).

In the context of the invention, it is understood as “biomarker” a characteristic that is objectively measured and evaluated as an indicator of normal biologic processes, pathogenic processes, or pharmacologic responses to a therapeutic intervention (NIH definition).

Therefore, biomarkers are used to indicate or measure a biological process (for instance, levels of a specific protein in blood or fluids, genetic mutations, or abnormalities observed in tests). Detecting biomarkers specific to a disease can aid in the identification, diagnosis, and treatment of affected individuals and people who may be at risk but do not yet exhibit symptoms.

Hence, another embodiment of the invention is the inflammatory skin diseases/disorders lesions biomarkers and/or gene expression products (including proteins) as biomarkers selected from the following:

Keratin 16 (KRT16); gap junction protein, beta 2, (connexin 26) (GJB2); chitinase 3-like 2 (CHI3L2); interleukin 8 (IL8); fatty acid binding protein 5 (FABP5); interleukin 1, beta (ID1B); signal transducer and activator of transcription (STAT1); heparanase (HPSE); solute carrier family 6 (amino acid transporter), member 14 (SLC6A14); transcobalamin I (vitamin B12 binding protein, R binder family) (TCN1); tumor necrosis factor (TNF); interleukin 1 family, member 5 (delta) (ID1F5); small proline-rich protein 2D (SPRR2D); kallikrein 13 (KLK13); chemokine (C-X-C motif) ligand 10 (CXCL10); desmoglein 3 (pemphigus vulgaris antigen) (DSG3); S100 calcium binding protein A12 (S100A12); interleukin 1 receptor antagonist (ID1RN); superoxide dismutase 2, mitochondrial (SOD2); keratin 6C; (KRT6E); interferon-induced protein with tetratricopeptide repeats 3 (IFIT3); desmocollin 2 (DSC2); endothelial cell growth factor 1 (platelet-derived) (ECGF1); RAS guanyl releasing protein 2 (calcium and DAG-regulated) (RASGRP2); wingless-type MMTV integration site family, member 5A (WNT5A); myxovirus (influenza virus) resistance 1, interferon-inducible protein p78 (mouse) (MX1); small proline-rich protein 1A (SPRR1A); defensin, beta 4 (DEFB4); S100 calcium binding protein A9 (S100A9); interleukin 1 family, member 9 (ID1F9); kallikrein 6 (neurosin, zyme) (KLK6); matrix metallopeptidase 9 (MMP9); serpin peptidase inhibitor, clade B (ovalbumin), member 3 (SERPINB3); interferon, gamma (IFNG); lipocalin 2 (oncogene 24p3) (LCN2); interferon, alpha-inducible protein 27 (IFI27); peroxisome proliferator-activated receptor delta (PPARD); serpin peptidase inhibitor, clade B (ovalbumin), member 1 (SERPINB1); latent transforming growth factor beta binding protein 1 (LTBP1); pre-B-cell colony enhancing factor 1 (PBEF1); transglutaminase 1 (K polypeptide epidermal type I, protein-glutamine-gamma-glutamyltransferase) (TGM1); chemokine (C-C motif) ligand 20 (CCL20); aldo-keto reductase family 1, member B10 (aldose reductase) (AKR1B10); S100 calcium binding protein A7 (S100A7).

In a specific embodiment, the invention relates to psoriatic scalp lesions biomarkers and/or gene expression products (including proteins) as biomarkers selected from the following: interleukin 8 (IL8); beta 4defensin (DEFB4); S100 calcium binding protein A7 (S100A7); S100 calcium binding protein A9 (calgranulin B) (S100A9); S100 calcium binding protein A12 (S100A12); interleukin 1b (IL-1b); lipocalin 2 (oncogene 24p3) (LCN2); transcobalamin I (vitamin B12 binding protein, R binder family) (TCN1); Interferon alpha-inducible protein 27 (IFI27); Peroxisome proliferator-activated receptor- [symbol] (PPAR-□); serpin peptidase inhibitor, clade B (ovalbumin), member 3 (SERPIN B3).

In another embodiment, the invention features an in vitro screening method of pharmacological agent/drug candidates (or family lead compound) susceptible of preventing and/or treating inflammatory skin diseases/disorders as well as scalp psoriasis, comprising determining the capacity of said pharmacological agent to modulate e. g. down regulated or up regulate) expression of said selected gene(s) expression and/or said biomarker (s)/gene expression product(s) levels or activity.

In a specific embodiment, the present invention features an in vitro screening method of drug candidates susceptible of preventing and/or treating inflammatory skin diseases/disorders; said method comprising the following steps:

Collecting at least two biological samples : one mimics pathological skin inflammatory lesion condition and the other mimics healthy condition;

Contacting at least one sample or a mixture of samples with one or more drug candidates to be tested;

Measuring gene expression or gene expression product level or activity in the biological samples or mixture obtained in b);

Selecting drug candidates which are capable of modulating gene expression or gene expression product level or activity measured in said samples or mixture obtained in b) and comparing the levels with a control sample, i.e., not mixed with drug candidate.

By “modulate” is understood any effect on expression or activity of biomarkers/gene expression products, any effect on genes or on activity of at least one of their expression promoter(s) and preferentially any effect inducing e. g. a down regulation or an up regulation, a stimulation, an inhibition, totally or partially.

In the context of the present invention, it is understood that <<expression of biomarkers/gene expression product>> refers to a quantity of a protein or any else product resulting from the transcription and/or translation of a gene. By “activity” is meant biological activity.

### BRIEF DESCRIPTION OF THE DRAWINGS AND TABLES

FIG. 1: RNA extraction from healthy volunteers and scalp psoriasis patients. a an active edge of psoriasis lesion on scalp; b hair follicles of telogen and anagen phases, with epithelial sheath intact (I), absent (II), or close to intact (III) c Representative chromatograms of micro-capillary electrophoresis of RNA collected from hair follicles of a healthy volunteer and a scalp psoriasis patient at three study visits (Baseline, Week 2 and 4).

FIG. 2: Total severity score (a) and Transcriptomic score (b) of patients at Baseline, Week 2 and Week 4 of clobetasol propionate shampoo treatment. (c) Average of transcriptomic score and TSS at Baseline, Week 2 and Week 4.

Table 1: List of 10 psoriasis disease-related genes that were significantly up-regulated in hair follicles of scalp psoriasis patients compared to healthy volunteers. Numbers in the column of “Fold increase in psoriasis skin” were extracted from Zhou X et al.¹⁸ except those of S100A7 and PPARδ, which were from Quekenborn-Trinquet V et al.¹⁹ Numbers in the column of “Fold increase in psoriasis hair follicles” and “p value” were results of this study. The p value of the significance of fold induction was calculated by t-test.

Table 2: Correlation from patients' clinical severity scores and transcriptomic score during clobetasol propionate shampoo treatment. For each clinical parameter, an “ok” variable was set to 1 if the clinical score and the transcriptomic score moved in the same direction, and set to 0 if the clinical score and the transcriptomic score moved in the opposite direction. The P value corresponded to the null hypothesis that the agreement probability P(ok=1) is ½.

Table 3: Fold modulation of 10 selected psoriasis disease-related genes in healthy volunteers and scalp psoriasis patients. For each gene, the average fold induction of gene expression in hair follicles of scalp psoriasis patients compared to healthy volunteers (psoriatic/healthy) was calculated. The transcriptomic score is the average fold induction of the 10 selected genes in each subject compared to the mean expression level in all healthy volunteers.

Table Supplementary 1: Quality and quantity of RNA collected from hair follicles of scalp psoriasis patients and healthy volunteers.

Table Supplementary S2: List of 44 genes reported to be associated to skin psoriasis and tested in TLDA in this study. Numbers in the column of “Fold increase in psoriasis skin” were extracted from Zhou X et al.,¹⁸ except those marked with *, which were from Quekenborn-Trinquet V et al.¹⁹ Numbers in the column of “Fold increase in psoriasis hair follicles” and “p value” were results of this study. p value was calculated by t-test.

In order to further illustrate the present invention and the advantages thereof, the following specific example is given, it being understood that same is intended only as illustrative and in nowise limitative. In said example to follow, all parts and percentages are given by weight, unless otherwise indicated.

### Example

### Non-invasive Gene Expression Profiling in Psoriatic Scalp Hair Follicles: Clobetasol Propionate Shampoo 0.05% Normalizes Psoriasis Disease Markers

The objective of this example is to determine whether psoriasis-related genes are differentially regulated in the hair follicles of scalp psoriasis patients and whether the modulation of these genes can be correlated with clinical severity scores.

Psoriasis is a common and chronic inflammatory disease estimated to affect about 2% of the Western population, with scalp being the most common site of involvement at the onset and throughout the course of the disease.¹ It is an immune-related disorder, triggered by activated T cells which infiltrate the dermis and stimulate hyperproliferation of keratinocytes.² Topical medication remains the most frequent treatment for scalp psoriasis in patients of all severity groups. Among the available treatments, clobetasol propionate shampoo was demonstrated to be effective and safe for patients with moderate or severe scalp psoriasis.³⁻⁶ It was designed to integrate a super potent corticosteroid (clobetasol propionate 0.05%) into a once-daily, short-contact shampoo formulation, in order to minimize the risk of adverse events associated with steroids usage, without compromising efficacy. It was also demonstrated that treatment with clobetasol propionate shampoo improved the patients' quality of life and resulted in high satisfaction⁷.

Large scale gene expression profiling has been widely used in the field of dermatology to elucidate the mechanisms of various diseases including psoriasis.⁸⁻⁹ Gene expression profiling using DNA microarray technology allows simultaneous examination of the expression levels of all human genes. Studies using peripheral blood mononuclear cells (PBMC) and skin samples harvested by biopsy or non-invasive tape striping technology identified genes which were differentially expressed in psoriatic and normal samples.¹⁰⁻¹³ Several of these genes, such as the S100 calcium-binding proteins and Defensins, mapped to known disease-associated loci and were previously shown to be up-regulated in psoriatic lesions.¹⁴⁻¹⁵ Recently identified new markers include genes involved in the Wnt pathway, disease-related cytokines and chemokines.¹⁶⁻¹⁸ Changes in disease-related gene expression levels were demonstrated to be correlated with stages of disease progression and clinical severity scores,¹⁹⁻²⁴ allowing it to be used for examining the effects of treatment. As examples, therapeutic antibodies against tumor necrosis factor alpha (TNFα) and interferon gamma (IFNγ), as well as the immune-modulatory drug pimecrolimus were shown to be effective in treating psoriasis.²⁵⁻²⁶

Although direct evidence is not available, several observations suggest that in scalp psoriasis patients, the hair follicles could be affected by the disease. First, although scalp psoriasis does not generally result in hair loss, extensive scarring alopecia can be induced in severely affected scalp areas.²⁷ Second, an irregular expansion in the proximal lower outer root sheath of hair follicles was observed in psoriatic biopsy samples, when compared to those of normal skin. This area, from which follicle regeneration occurs during the anagen phase of hair growth, was considered as putative stem cell region due to the presence of keratin 19-containing cells.²⁸ Finally, when the growth kinetics of anagen hair follicles was measured using DNA flow-cytometry, the infundibular portion of the psoriatic hair sample demonstrated a significant increase of proliferation activity in S-phase compared to samples from healthy scalps.²⁹

It was hypothesized that scalp psoriasis does not only affect hair follicle growth parameters, but also leads to modulation of psoriasis disease-related genes. To test this hypothesis, a medium scale gene expression profile was generated using RNA extracted from hair follicles of scalp psoriasis patients and healthy volunteers. A study was conducted of the effect of clobetasol propionate shampoo treatment on regulation of these genes and to correlate the disease-related gene expression levels with clinical severity scores of scalp psoriasis patients.

Methods and Materials:

This study was conducted in accordance with the Declaration of Helsinki, its amendments, Good Clinical Practice and local regulatory requirements including ethics board review. All patients provided written informed consent before entering into the study.

Study Design and Patient Selection:

This study was part of a single arm, open study, which comprised the preliminary phase of a double-blind, multi-centre and controlled study on the maintenance effect of clobetasol propionate shampoo. 59 patients were recruited in three centers of Canada for this part of the study. The recruited patients were 18 years or older, with “moderate” or “severe” scalp psoriasis based on their Global Severity Score (GSS) assessment (GSS=3 or 4 on a scale of 0 to 5, with 0=clear and 5=very severe).

Treatment and Clinical Assessments:

All patients received clobetasol propionate shampoo 0.05% (Clobex® shampoo, Galderma Laboratories, LP, Fort Worth, Tex., U.S.A) for up to 4 weeks. The study drug was applied once daily by patients in a thin film onto dry affected scalp areas and left in place for 15 minutes before lathering and rinsing.

The study visits were conducted at Baseline, Week 2 and Week 4. At each visit, the investigators assessed various clinical severity parameters, including erythema (E), scaling (S), plaque thickening (P) (all on a scale of 0 to 4, with 0=none and 4=very severe), extent of disease (Ex) (on a scale of 0 to 5, with 0=none and 5=80-100%) and GSS. Patients were also asked to indicate their level of pruritus (on a scale of 0 to 3, with 0=none to 3=severe) at each visit. At the end of study, Total Severity Score (TSS) was calculated as TSS=E+S+P, and Modified Psoriasis Area and Severity Index (MPASI) was calculated as MPASI=(E+S+P)*Ex.³⁰

Sample Collection and RNA Exaction:

Hair samples were collected from the recruited patients at each study visit. A minimum of 15 anagen phase hair follicles were plucked from the active edge of psoriatic lesions. The hair follicles with a bulb and an intact or close-to-intact sheath were subsequently processed. Plucked anagen hair follicles from 8 healthy volunteers were also included for analysis.

Hair shafts were cut 1-2 mm above the dermal sheath and dissolved in 500 μl RLT buffer (Qiagen Inc.) with 10 μl/ml β-mercaptoethanol. Total RNA was extracted using RNeasy extraction kits (Qiagen Inc.) according to manufacturer's protocol. RNA Quantity was measured using Quant-it RNA assay kit (Molecular Probes) and the quality was monitored by following the electrophoresis behavior of RNA using a 2100 Bioanalyser (Agilent). 50 ng of extracted RNA of good quality [RNA indication number (RIN)≧7] and a minimum concentration of 4 ng/μl was then used for synthesizing cDNA using high capacity cDNA archive kits (Applied Biosystems).

TaqMan Low Density Array (TLDA) Analysis:

A single TLDA array contains 8 replicates of the PCR primers for 48 genes (44 selected genes of interest and 4 housekeeping genes). A single determination was performed for samples from scalp psoriasis patients (SP), while samples from healthy volunteers (HV) were analyzed in duplicates.

Synthesized cDNA was added to the PCR master mix, and the mixture was loaded by centrifugation into the wells of the array containing the lyophilized primer sets (Applied Biosystems). The wells were sealed and the reactions were conducted on ABI 7900HT (Applied Biosystems). PCR threshold cycle (Ct) numbers at which the fluorescent signal of the generated nascent DNA exceeds a threshold value was determined. The Ct number was normalized by first subtracting the average Ct of the housekeeping genes in the same sample, and then adding back the average Ct of the housekeeping genes across all samples.

Statistical Analysis:

The fold modulation of gene expression of scalp psoriasis samples versus samples of healthy volunteers was defined as 2^{(mean CtHV-mean CtSP)}, with Ct_{HV} and Ct_{SP} depicting the Ct values of healthy volunteer and scalp psoriasis samples, respectively. One-way ANOVA with Benjamini-Hochberg multiplicity correction was performed using JMP 7.0.1 (SAS Institute) and irMF 3.5 (National Institute of Statistical Sciences, NISS) software, in order to identify genes that were significantly modulated in scalp psoriasis samples.

To assess the correlation from the transcriptomic score and clinical severity scores, an “ok” variable was created and defined as follows: the variable was set to 1 if transcriptomic score and clinical score change toward the same direction; otherwise the variable was set to 0. The p value of the analysis corresponded to the null hypothesis that the agreement probability Pₒₖ₌₁ is 0.5.

Results:

Inflammation-Related Genes Are Up-Regulated in Scalp Hair Follicles of Psoriasis Patients:

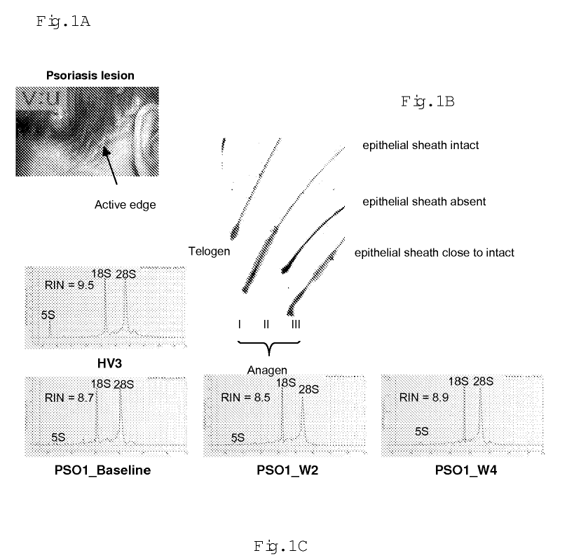

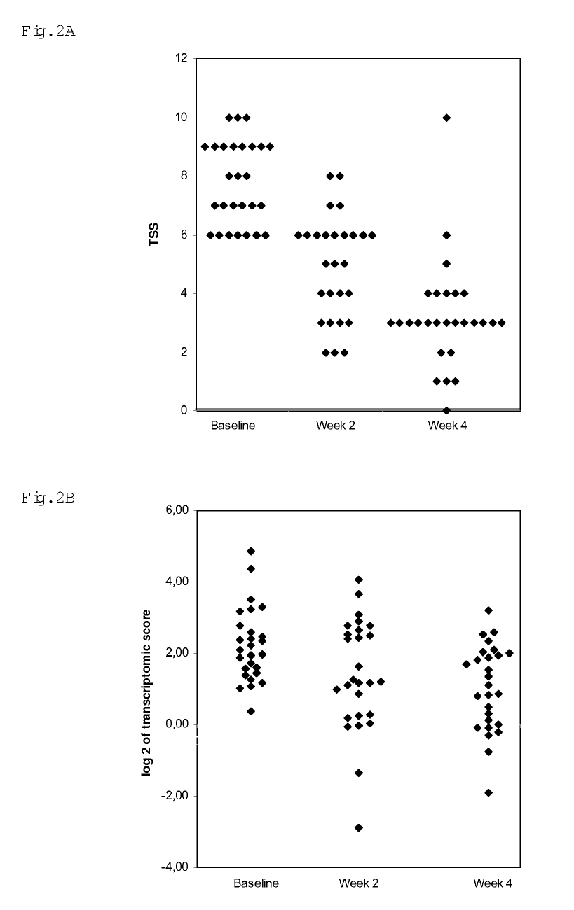

To determine whether hair follicles are affected by scalp psoriasis, gene expression profiles were generated using RNA extracted from hair follicles of both scalp psoriasis patients and healthy volunteers. Hair samples were collected at Baseline, Week 2 and 4 of treatment with clobetasol propionate shampoo. A minimum of 15 anagen phase hair follicles were plucked from the active edge of psoriatic lesions (FIG. 1a). Only hair follicles with a bulb and an intact or close-to-intact sheath were processed (FIG. 1b, I and III). Hair plucking caused only mild discomfort to patients and volunteers, and did not induce Koebner phenomenon when performed at sites of remission. Quality of RNA extracted from hair follicles was evaluated by micro-capillary electrophoresis and representative chromatograms are shown in FIG. 1c. For all RNA samples, the 18S, 28S and 5S ribosomal RNA peaks were clearly visible, with no degradation detected. RIN, an indicator of RNA quality, was calculated for each sample. Extracted RNA from all 8 healthy volunteers and from 31 of 59 patients had a RIN of 7 or higher, adequate for RT-PCR analysis.³¹ The concentration of extracted RNA was variable among samples, but nevertheless all fulfilled the minimum requirement for the downstream procedure (Table S1). Taken together, we obtained RNA of good quality and sufficient quantity for gene expression analysis from both healthy volunteers and scalp psoriasis patients.

The RNA extracted from volunteers and patients was subsequently used for TLDA analysis, a high through-put functional genomics screening technology.³²⁻³⁴ In scalp hair follicles, the expression levels of 44 genes were determined that were previously reported to be up-regulated in psoriasis skin lesions (Table S2). Four housekeeping genes (18S rRNA, β-actin, GAPDH and HPRT1) were also included in the analysis for normalization purposes. Among the 31 samples whose RNA quality and quantity were adequate for TLDA analysis, 28 samples generated data of good quality based on the expression levels of housekeeping genes and were proceeded to statistical analysis. A total of 10 genes were determined to be significantly up-regulated in hair follicles of scalp psoriasis patients compared to healthy volunteers (≧1.8 fold induction on average with p≦0.01) (Table 1). These 10 genes were reported to be modulators of the inflammatory response, or to be up-regulated under inflammatory conditions, indicating that the hair follicles of scalp psoriasis patients were affected by inflammation.

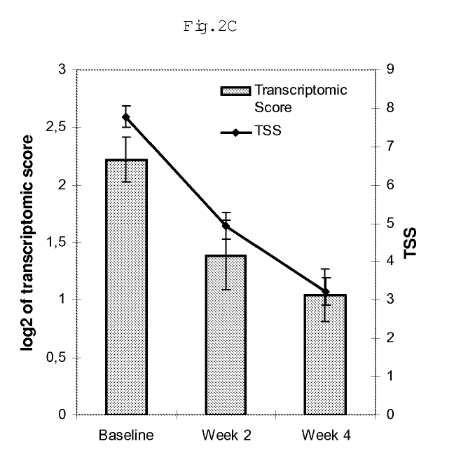

The heat map showing the modulation of the 10 genes is depicted in FIG. 2. The genes were arranged from left to right according to the average fold induction of expression level in hair follicles of scalp psoriasis patients versus healthy volunteers. To arrange subjects, a transcriptomic score was defined as the average fold induction of the gene expression level in each subject compared to the mean level in all healthy volunteers. When the subjects were arranged based on their transcriptomic scores, all scalp psoriasis patients had a score equal to or higher than 2 and clustered in a distinct group, except one patient which was inserted among the healthy volunteers, indicating that the transcriptomic score can be considered as a molecular indicator of disease severity.

Clobetasol Propionate Shampoo Is Effective in Decreasing Both Transcriptomic Score and Severity of Scalp Psoriasis:

Recruited scalp psoriasis patients received daily treatment with clobetasol propionate shampoo, and the effect of treatment was evaluated by transcriptomic score and various clinical assessments. After 4 weeks of daily treatment, the mean GSS decreased from 3.5±0.5 to 1.8±0.8, the mean MPASI decreased from 21.1±12.3 to 5.3±6.0 and the mean TSS decreased from 7.8±1.4 to 3.2±1.8 (FIG. 3a). Correspondingly, pruritus, extent of the disease and individual sign scores including erythema, scaling and plaque thickening improved after treatment (data not shown). The transcriptomic score decreased after 2 or 4 weeks of treatment as well (FIG. 3b). Therefore, the treatment of clobetasol propionate shampoo induced a strong and progressive decrease in both transcriptomic score and clinical severity score such as TSS (FIG. 3c), suggesting that the treatment was effective not only in improving the scalp psoriasis lesion conditions, but also in relieving the inflammatory response.

To determine whether the transcriptomic score and clinical severity scores are correlated, it was examined whether these scores change towards the same direction upon treatment with clobetasol propionate shampoo. As shown in Table 2, skin phototype as an intrinsic parameter of each patient, remained unchanged upon treatment and therefore was not correlated with the transcriptomic score. Neither was extent of the disease correlated with transcriptomic score during the treatment. However, the other clinical severity scores examined, including MPASI, GSS, TSS, pruritus, erythema, scaling and plaque thickening, all demonstrated a significant correlation after 4-weeks of treatment. The correlation was strong but less significant when assessed after two weeks of treatment. Taken together, these results suggested that the transcriptomic score is a suitable molecular and local indicator for the clinical severity of scalp psoriasis.

For the first time and as shown in FIG. 2, it is demonstrated that scalp psoriasis is associated to significant effects on gene expression in plucked hair follicles. So, hair follicles collected by a non-invasive method can be used to monitor skin or scalp inflammation.

Using the predictive model, the effects of Clobex shampoo 0.05% was monitored on a molecular level, by a non-invasive method in hair follicles of the psoriatic scalp.

As shown in FIG. 3, the clobex shampoo efficiency is indicated following expression of 12 discriminating genes in hair follicles of the psoriatic scalp collected by a non-invasive method.

Discussion:

In the present study, it was demonstrated that 10 inflammation-related genes were significantly up-regulated in the hair follicles of scalp psoriasis patients. The transcriptomic score was defined as the mean fold modulation of the expression level of the 10 genes, showed that the score indicated severity of the disease on a molecular level and that it correlated with various clinical assessments, including GSS, TSS and MPASI. Clobetasol propionate shampoo treatment, which was demonstrated to be effective in treatment of the clinical signs of scalp psoriasis, also led to a decrease of the transcriptomic score.

Although studies using skin biopsy have generated valuable information on psoriasis, the invasive nature of the technique render it impractical as a routine method for monitoring the disease progression or pharmacogenomic effects of various treatments. A tape-striping method was developed and utilized to harvest RNA for gene expression profiling; however, it has the drawbacks of requiring large skin surface areas and produces only low RNA yield, particularly in healthy volunteers.^{13, 35-36} This study validated that hair follicle plucking is suitable for collecting RNA of good quality and sufficient quantity for gene expression profiling. Therefore, this minimally invasive technique can be used to diagnose scalp psoriasis and to study the mechanism of the disease.

It was demonstrated for the first time that the scalp hair follicle cells were affected by psoriasis. The 44 genes, whose expression level in hair follicles was examined in this study, were selected based on their reported elevated expression level in skin or blood samples harvested from psoriasis patients.¹⁸⁻¹⁹ These genes were known to be involved in various biological pathways, including inflammation, immune response, proliferation and differentiation of the epidermis. The 10 genes, which were demonstrated to be significantly over-expressed in hair follicles of scalp psoriasis patients, are all functionally related to inflammation. They either act as regulators or mediators of inflammation (IL-8,³⁷ LCN2,³⁸ PBEF1,³⁹ HPSE,⁴⁰ DEFB4⁴¹ and three members of the S100 protein family, S100A7,⁴² S100A9⁴³ and S100A12⁴⁴) or are up-regulated under inflammatory conditions (IF127).⁴⁵ PPARδ was demonstrated to be involved in both modulation of inflammation and proliferation of keratinocytes.⁴⁶ It was also shown to enhance keratinocyte proliferation in psoriasis.⁴⁷ Based on the reported functions and the observed up-regulation of these genes, it was concluded that the hair follicles of scalp psoriasis patients are affected by inflammation. Clobetasol propionate shampoo was demonstrated to be effective and safe in treatment of moderate to severe scalp psoriasis, since its usage results in lower scores of individual signs and global assessments.³⁻⁶ In this study, it was demonstrated that clobetasol propionate shampoo also led to a decrease of patients' transcriptomic scores, thus the treatment down-regulated the genes which were over-expressed under psoriasis conditions. Since all these genes play a role in inflammation, this result strongly suggests that clobetasol propionate shampoo was effective in alleviating the signs of inflammation in scalp hair follicles, confirming the previously reported anti-inflammatory property of corticosteroids.⁴⁸ Furthermore, it implicates that the clinical symptoms of scalp psoriasis are at least in part caused by inflammation.

It was observed that the transcriptomic score correlated with clinical severity of scalp psoriasis, based on the difference of transcriptomic score among healthy volunteers and psoriasis patients, and on the improvement of both disease severity and transcriptomic score upon clobetasol propionate treatment. However, it should be noted that the present study is based on the analysis of a restricted set of genes previously identified in skin biopsies. Disease markers of psoriatic skin might not be the best choice for genes to be followed in scalp psoriasis; it is therefore likely that a better correlation can be achieved by generating a large scale gene expression profile to identify robust biomarkers of scalps psoriasis, whose change of expression level is then followed. Furthermore, since hair plucking at study visits was not guided with precise localization technique and was instead conducted always at the active edge of psoriatic lesions, it is possible that a slightly different region was sampled each time due to the decrease of disease extent throughout treatment (data not shown). In future studies, precise localization of the plucked areas, as well as local clinical scoring techniques should further improve the correlation from clinical and molecular severity parameters.

Being a local indication, transcriptomic score has its limitation when compared to clinical parameters, which are global assessments. Consistently, the transcriptomic score correlates significantly with individual sign scores, TSS, GSS and MPASI, but not with extent of the disease. Image-guided hair sampling in several different scalp locations could constitute a solution to this limitation and lead to a more generalized transcriptomic score, which would reflect not only the local clinical severity, but also the extent of the disease.

As costs for development of new drugs rise constantly, while chances of success stagnate, initiatives were launched in the U.S. and Europe calling for the development of new tools including biomarkers, that make the drug development process more efficient and effective (The critical path initiative [http://www.fda.gov/oc/initiatives/criticalpath/]; Innovative Medicines Initiative [http://imi.europa.eu/index_en.html]). Furthermore, rules for exploratory investigational new drugs studies have been issued (http://www.fda.gov/cder/guidance/7086fnl.htm). These studies usually involve very limited human exposure and have no therapeutic intent; however, they can determine whether a mechanism of action defined in experimental systems can also be observed in humans, thereby allowing early decision-taking in the development process. The results described in this article set the basis for applying genomic biomarker studies on tiny skin surfaces to test the efficacy of drugs for the treatment of scalp psoriasis.

Thus the present example demonstrates that the RNA of good quality and sufficient quantity was obtained from hair follicles of psoriasis patients and healthy volunteers. The expression level of 10 inflammation-related genes was significantly increased in psoriatic hair follicles. The patient's transcriptomic score, defined as the mean fold modulation of these 10 genes compared to healthy volunteers, correlated with clinical severity scores. Clobetasol propionate shampoo was effective in decreasing both the transcriptomics and the clinical severity scores.

Hence, hair follicles of scalp psoriasis patients are affected by the inflammatory process. The change of the expression level of inflammation-related genes correlates with the severity of the disease.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

### REFERENCES

1. Van de Kerkhof P C, Franssen M E. Psoriasis of the scalp. Diagnosis and management. Am J Clin Dermatol 2001; 2:159-165.
2. Lowes M A, Bowcock A M, Krueger J G. Pathogenesis and therapy of psoriasis. Nature 2007; 445:866-73.
3. Jarratt M, Breneman D, Gottlieb A B et al. Clobetasol propionate shampoo 0.05%: a new option to treat patients with moderate to severe scalp psoriasis. J Drugs Dermatol 2004; 3:367-373.
4. Andres P, Poncet M, Sidou F, Soto P. Short-term safety assessment of clobetasol propionate 0.05% shampoo: hypothalamic-pituitary-adrenal axis suppression, atrophogenicity and ocular safety in subjects with scalp psoriasis. J Drugs Dermatol 2006; 5:328-332.
5. Reygagne P, Mrowietz U, Decroix J et al. Clobetasol propionate shampoo 0.05% and calcipotriol solution 0.005%: a randomized comparison of efficacy and safety in subjects with scalp psoriasis. J Dermatol Treat 2005; 16:31-36.
6. Griffiths C E M, Finlay A Y, Fleming C J et al. A randomized, investigator-masked clinical evaluation of the efficacy and safety of clobetasol propionate shampoo 0.05% shampoo and tar blend 1% shampoo in the treatment of moderate to severe scalp psoriasis. J Dermatol Treat 2006; 17:90-95.
7. Tan J, Thomas R, Wang B et al. Treatment of scalp psoriasis with short-contact clobetasol propionate shampoo 0.05% results in high patient satisfaction and quality of life improvement. Submitted to JDD.
8. Kunz M, Ibrahim S M, Koczan D et al. DNA microarray technology and its applications in dermatology. Exp Dermatol 2004; 13:593-606.
9. Kunz M. DNA microarray technology in dermatology. Semin Cutan Med Surg 2008; 27:16-24.
10. Bowcock A M, Shannon W, Du F et al. Insights into psoriasis and other inflammatory diseases from large-scale gene expression studies. Hum Mol Genet 2001; 10:1793-805.
11. Oestreicher J L, Walters I B, Kikuchi T et al. Molecular classification of psoriasis disease-associated genes through pharmacogenomic expression profiling. Pharmacogenomics J 2001; 1:272-87.
12. Wong R, Tran V, Morhenn V et al. Use of RT-PCR and DNA microarrays to characterize RNA recovered by non-invasive tape harvesting of normal and inflamed skin. J Invest Dermatol 2004; 123:159-67.
13. Benson N R, Papenfuss J, Wong R et al. An analysis of select pathogenic messages in lesional and non-lesional psoriatic skin using non-invasive tape harvesting. J Invest Dermatol 2006; 126:2234-41.
14. Hards BD, Zhao X, Zhang J et al. Assignment of psoriasin to human chromosomal band 1q21: Co-ordinate overexpression of clustered genes in psoriasis. J Invest Dermatol 1996; 106:753-8.
15. Schroder J M, Harder J. Human beta-defensin-2. Int J Biochem cell Biol 1999; 31:645-51.
16. Koczan D, Guthke R, Thiesen H J et al. Gene expression profiling of peripheral blood mononuclear leukocytes from psoriasis patients identifies new immune regulatory molecules. Eur J Dermatol 2005; 15:251-7.
17. Reischl J, Schwenke S, Beekman J M et al. Increased Expression of Wnt5a in Psoriatic Plaques. J Invest Dermatol 2007; 127:163-9.
18. Zhou X, Krueger J G, Kao M C et al. Novel mechanisms of T-cell and dendritic cell activation revealed by profiling of psoriasis on the 63,100-element oligonucleotide array. Physiol Genomics 2003; 13:69-78.
19. Quekenborn-Trinquet V, Fogel P, Aldana-Jammayrac O et al. Gene expression profiles in psoriasis: analysis of impact of body site location and clinical severity. Br J Dermatol 2005; 152:489-504.
20. Bonifati C, Carducci M, Cordiali P et al. Correlated increase of tumor necrosis factor-α, interleukin-6 and granulocyte monocytecolony stimulating factor levels in suction blister fluids and sera of psoriatic patients—relationship with disease severity. Clin Exp Dermatol 1994; 19:383-7.
21. Alkemade H, de Jongh G, Arnold P et al. Levels of skin-derived antileukoproteinase (SKALP)/elafin in serum correlate with disease activity during treatment of severe psoriasis with cyclosporine A. J Incest dermatol 1995; 104:189-93.
22. De pita O, Frezzolini A, Cianetti A et al. Squamous cell carinoma-related antigen (SCCr-Ag), sICAM-1 and beta-2microglobulin are useful markers of disease activity in psoriasis. Acta Derm Venereol (Stockh) 1999; 79:132-5.
23. Szepietowski J, Wasik F, Bielicka E et al. Soluble E-selectin serum levels correlate with disease activity in psoriatic patients. Clin Exp Dermatol 1999; 24:33-6.
24. lnaoki M, Sato S, Shimada Y et al. Decreased expression levels of L-selectin on subsets of leucocytes and increased serum L-selectin in severe psoriasis. Clin Exp Immunol 2000; 122:484-92.
25. Haider A S, Cohen J, Fei J et al. Insights into gene modulation by therapeutic TNF and IFNgamma antibodies: TNF regulates IFNgamma production by T cells and TNF-regulated genes linked to psoriasis transcriptome. J Invest Dermatol 2008; 128:655-66.
26. Rappersberger K, Komar M, Ebelin M E et al. Pimecrolimus identifies a common genomic anti-inflammatory profile, is clinically highly effective in psoriasis and is well tolerated. J Invest Dermatol 2002; 119:876-87.
27. Bardazzi F, Fanti P A, Orlandi C et al. Psoriatic scarring alopecia: observations in four patients. Int J Dermatol 1999; 38:765-8.
28. Wilson C L, Dean D, Lane E B et al. Keratinocyte differentiation in psoriatic scalp: morphology and expression of epithelial keratins. Br J Dermatol 1994; 131:191-200.
29. Katsuoka K, Schell H, Deinlein E, Hornstein O P. Cell kinetics of the human anagen hair follicle. Flow cytometric studies in healthy and psoriatic subjects. Dermatologica 1987; 174:105-9.
30. Wulff-woesten A, Ohlendorf D, Henz B M, Haas N. Dithranol is an emulsifying oil base (bio-wash-oil) for the treatment of psoriasis of the scalp. Skin Pharmacol Physiol 2004; 17:91-7.
31. Fleige S, Pfaffl M W. RNA integrity and the effect on the real-time qRT-PCR performance. Mol Aspects Med 2006; 27:126-39.
32. Chiu S T, Hsieh F J, Chen S W et al. Clinicopathologic correlation of up-regulated genes identified using cDNA microarray and real-time reverse transcription-PCR in human colorectal cancer. Cancer Epidemiol Biomarkers Prev 2005; 14:437-43.
33. Lü B, Xu J, Chen J et al. TaqMan low density array is roughly right for gene expression quantification in colorectal cancer. Clin Chim Acta 2008; 389:146-51.
34. Goulter A B, Harmer D W, Clark K L. Evaluation of low density array technology for quantitative parallel measurement of multiple genes in human tissue. BMC Genomics 2006; 7:34.
35. Wong R, Tran V, Morhenn V et al. Use of RT-PCR and DNA microarrays to characterize RNA recovered by non-invasive tape harvesting of normal and inflamed skin. J Invest Dermatol 2004; 123:156-67.
36. Wong R, Tran V, Talwalker S, Benson N R. Analysis of RNA recovery and gene expression in the epidermis using non-invasive tape stripping. J Dermatol Sci 2006; 44:81-92.
37. Pietrzak A T, Zalewska A, Chodorowska G et al. Cytokines and anticytokines in psoriasis. Clin Chim Acta 2008; 394:7-21.
38. Kjeldsen L, Johnsen A H, Sengeløv H, Borregaard N. isolation and primary structure of NGAL, a novel protein associated with human neutrophil gelatinase. J Biol Chem 1993; 268:10425-32.
39. Luk T, Malam Z, Marshall J C. Pre-B cell colony-enhancing factor (PBEF)/visfatin: a novel mediator of innate immunity. J Leukoc Biol 2008; 83:804-16.
40. McKenzie E A. Heparanase: a target for drug discovery in cancer and inflammation. Br J Pharmacol 2007; 151:1-14.
41. Yamasaki K, Gallo R L. Antimicrobial peptides in human disease. Eur J Dermatol 2008; 18:11-21.
42. Zheng Y, Niyonsaba F, Ushio H et al. Microbicidal protein psoriasin is a multifunctional modulator of neutrophil activation. Immunology 2008;
43. Gebhardt C, Németh J, Angel P, Hess J. S100A8 and S100A9 in inflammation and cancer. Biochem Pharmacol 2006; 72:1622-31.
44. Donato R. RAGE: a single receptor for several ligands and different cellular responses: the case of certain S100 proteins. Curr Mol Med 2007; 7:711-24.
45. Suomela S, Cao L, Bowcock A, Saarialho-Kere U. Interferon alpha-inducible protein 27 (IFI27) is upregulated in psoriatic skin and certain epithelial cancers. J Invest Dermatol 2004; 122:717-21.
46. Michalik L, Wahli W. Peroxisome proliferator-activated receptors (PPARs) in skin health, repair and disease. Biochim Biophys Acta 2007; 1771:991-8.
47. Romanowska M, al Yacoub N, Seidel H et al. PPARdelta enhances keratinocyte proliferation in psoriasis and induces heparin-binding EGF-like growth factor. J Invest Dermatol 2008; 128:110-24.
48. Morand E F. Effects of glucocorticoids on inflammation and arthritis. Curr Opin Rheumatol 2007; 19:302-7.

TABLE 1

| | | | Fold | Fold | |
|---|---|---|---|---|---|
| | | | increase in | increase in | |
| | | | psoriasis | psoriasis | p |
| Gene | Name | Function | skin | hair follicles | value |
| IL8 | interleukin 8 | inflammatory response/ | 13.9 | 29.5 | <0.001 |
| | | chemotaxis | | | |
| DEFB4 | defensin, beta 4 | immune response/defense | 5.7 | 20.3 | <0.001 |
| | | response | | | |
| S100A7 | S100 calcium | epidermis development/ | 296 | 11.1 | <0.001 |
| | binding protein A7 | angiogenesis | | | |
| S100A9 | S100 calcium | inflammatory response | 3.1 | 6.3 | <0.001 |
| | binding protein A9 | | | | |
| | (calgranulin B) | | | | |
| S100A12 | S100 calcium | inflammatory response | 33.6 | 5.0 | 0.003 |
| | binding protein A12 | | | | |
| | (calgranulin C) | | | | |
| LCN2 | lipocalin 2 (oncogene | modulator of inflammation | 9.9 | 2.0 | 0.001 |
| | 24p3) | | | | |
| IFI27 | interferon, alpha- | ND | 3.5 | 1.9 | 0.002 |
| | inducible protein 27 | | | | |
| PBEF1 | pre-B-cell colony | positive regulation of cell | 3.4 | 1.8 | 0.004 |
| | enhancing factor 1 | proliferation/cell-cell | | | |
| | | signalling | | | |
| HPSE | heparanase | proteoglycan metabolic | 4.9 | 1.8 | 0.01 |
| | | process | | | |
| PPARδ | peroxisome | fatty acid catabolic and | 5.5 | 1.8 | 0.01 |
| | proliferator-activated | epidermis proliferation | | | |
| | receptor delta | | | | |

TABLE 2

| Baseline → Week 4 | | | Baseline → Week 2 | | |
|---|---|---|---|---|---|
| | # ok | | | # ok | |
| | (total = | | | (total = | |
| Parameter | 27) | P value | Parameter | 27) | P value |
| GSS | 22 | 0.0002 | GSS | 20 | 0.0030 |
| MPASI | 22 | 0.0002 | MPASI | 17 | 0.0610 |
| TSS | 22 | 0.0002 | TSS | 17 | 0.0610 |
| Erythema | 22 | 0.0002 | Erythema | 18 | 0.0261 |
| Scaling | 22 | 0.0002 | Scaling | 21 | 0.0008 |
| Plaque | 22 | 0.0002 | Plaque | 17 | 0.0610 |
| thickening | | | thickening | | |
| Pruritus | 19 | 0.0096 | Pruritus | 17 | 0.0610 |
| Extent of | 16 | 0.1239 | Extent of | 13 | 0.5000 |
| disease | | | disease | | |
| Skin phototype | 6 | 0.9970 | Skin phototype | 9 | 0.9390 |

TABLE 3

| | | | | | | | | | | | Transcriptomic |
|---|---|---|---|---|---|---|---|---|---|---|---|
| Sample | IL8 | DEFB4 | S100A7 | S100A9 | S100A12 | LCN2 | IFI27 | PBEF1 | HPSE | PPARd | score |
| HV1 | 0.1 | 0.3 | 0.7 | 0.6 | 0.5 | 0.7 | 0.9 | 0.8 | 0.9 | 1.1 | 0.6 |
| HV2 | 0.1 | 0.5 | 0.9 | 0.5 | 0.3 | 1.3 | 0.8 | 1.2 | 0.9 | 1.2 | 0.6 |
| HV3 | 0.7 | 0.3 | 0.8 | 0.7 | 0.6 | 1.0 | 1.2 | 1.0 | 0.7 | 1.2 | 0.8 |
| HV4 | 1.5 | 0.4 | 1.0 | 0.6 | 0.8 | 0.8 | 1.3 | 1.1 | 0.5 | 1.5 | 0.9 |
| HV5 | 2.5 | 2.1 | 0.8 | 0.9 | 0.9 | 0.6 | 0.9 | 1.0 | 0.9 | 0.9 | 1.0 |
| HV6 | 1.4 | 2.0 | 0.9 | 1.7 | 1.2 | 1.0 | 0.5 | 1.0 | 1.1 | 0.7 | 1.1 |
| PSO22 | 0.1 | 3.8 | 5.3 | 1.5 | 1.5 | 0.9 | 1.5 | 1.3 | 0.8 | 1.8 | 1.3 |
| HV7 | 1.7 | 2.8 | 1.2 | 3.0 | 5.7 | 1.4 | 1.5 | 1.1 | 2.3 | 0.6 | 1.8 |
| HV8 | 8.9 | 4.9 | 2.2 | 1.5 | 2.7 | 1.4 | 1.4 | 0.9 | 1.5 | 1.0 | 2.0 |
| PSO5 | 3.4 | 2.3 | 3.3 | 3.1 | 1.0 | 2.5 | 2.0 | 1.0 | 1.3 | 2.3 | 2.0 |
| PSO20 | 1.9 | 3.0 | 4.0 | 2.0 | 2.0 | 2.0 | 2.3 | 1.5 | 1.5 | 1.8 | 2.1 |
| PSO6 | 14.2 | 3.2 | 5.2 | 1.9 | 0.8 | 2.6 | 2.7 | 1.1 | 0.7 | 1.6 | 2.3 |
| PSO26 | 17.6 | 7.5 | 5.7 | 4.3 | 1.7 | 1.2 | 0.9 | 1.1 | 1.1 | 1.0 | 2.4 |
| PSO2 | 6.4 | 1.7 | 13.7 | 6.7 | 2.6 | 1.9 | 1.4 | 1.4 | 0.9 | 1.7 | 2.6 |
| PSO28 | 22.6 | 22.2 | 6.9 | 3.3 | 0.7 | 1.2 | 1.9 | 1.2 | 1.4 | 0.7 | 2.7 |
| PSO8 | 7.6 | 6.7 | 7.6 | 4.0 | 7.4 | 0.9 | 1.2 | 1.3 | 1.1 | 3.0 | 3.0 |
| PSO13 | 11.3 | 13.3 | 9.1 | 4.3 | 0.9 | 2.1 | 1.8 | 1.5 | 1.4 | 1.6 | 3.0 |
| PSO15 | 3.1 | 3.3 | 7.7 | 2.9 | 14.3 | 1.8 | 1.7 | 1.2 | 1.9 | 3.7 | 3.1 |
| PSO27 | 1.7 | 14.1 | 10.1 | 6.2 | 3.3 | 1.9 | 2.3 | 1.4 | 3.0 | 1.5 | 3.3 |
| PSO3 | 6.8 | 5.4 | 13.8 | 10.0 | 3.0 | 3.5 | 1.8 | 1.6 | 2.0 | 1.4 | 3.6 |
| PSO7 | 71.3 | 2.9 | 8.6 | 7.7 | 3.6 | 1.8 | 1.2 | 1.4 | 1.1 | 4.0 | 3.8 |
| PSO10 | 194.4 | 10.1 | 7.6 | 2.7 | 1.1 | 1.6 | 2.8 | 1.4 | 1.8 | 1.6 | 3.9 |
| PSO17 | 34.5 | 32.3 | 13.0 | 6.4 | 4.1 | 0.9 | 3.4 | 1.9 | 3.4 | 0.3 | 4.3 |
| PSO11 | 25.1 | 9.3 | 11.7 | 7.1 | 5.8 | 1.7 | 2.2 | 2.4 | 1.5 | 3.3 | 4.7 |
| PSO25 | 34.6 | 93.0 | 7.8 | 4.8 | 7.1 | 1.2 | 1.8 | 1.1 | 2.6 | 1.8 | 5.0 |
| PSO24 | 40.2 | 11.0 | 18.4 | 6.4 | 3.9 | 3.3 | 2.6 | 1.5 | 2.5 | 1.9 | 5.1 |
| PSO1 | 16.6 | 67.3 | 26.4 | 9.5 | 8.1 | 2.4 | 1.0 | 2.1 | 1.4 | 1.1 | 5.3 |
| PSO14 | 286.0 | 8.9 | 15.1 | 4.1 | 4.3 | 2.3 | 2.1 | 1.6 | 3.0 | 1.5 | 5.5 |
| PSO16 | 1.9 | 12.3 | 21.6 | 25.9 | 10.7 | 4.8 | 4.2 | 4.0 | 1.0 | 5.8 | 6.0 |
| PSO9 | 31.2 | 116.9 | 28.4 | 10.8 | 10.2 | 3.2 | 1.5 | 1.9 | 1.3 | 1.6 | 6.8 |
| PSO19 | 174.9 | 86.6 | 15.4 | 14.1 | 9.5 | 5.1 | 2.0 | 2.2 | 2.3 | 2.1 | 9.0 |
| PSO12 | 817.6 | 375.3 | 18.3 | 10.9 | 13.2 | 0.6 | 0.5 | 5.3 | 1.7 | 2.4 | 9.4 |
| PSO18 | 612.2 | 121.5 | 16.3 | 14.0 | 14.7 | 4.2 | 1.7 | 2.2 | 2.2 | 0.9 | 9.8 |
| PSO4 | 151.4 | 404.8 | 16.9 | 11.1 | 40.8 | 2.3 | 2.0 | 2.3 | 4.4 | 1.9 | 11.5 |
| PSO21 | 15856.8 | 775.8 | 17.4 | 16.9 | 21.8 | 1.8 | 1.8 | 9.6 | 2.8 | 2.2 | 20.7 |
| PSO23 | 1390.2 | 729.6 | 26.9 | 27.4 | 324.7 | 2.8 | 6.2 | 4.3 | 10.3 | 2.3 | 29.1 |
| psoriatic/ | 29.5 | 20.3 | 11.1 | 6.3 | 5.0 | 2.0 | 1.9 | 1.8 | 1.8 | 1.8 | |
| healthy | | | | | | | | | | | |

TABLE SUPPLEMENTARY 1

| | RIN | | | Concentration (ng/μl) | | |
|---|---|---|---|---|---|---|
| | | Clobex daily | | | Clobex daily | |
| | Baseline | treatment | | Baseline | treatment | |
| Sample | (W0) | Week 2 | Week 4 | (W0) | Week 2 | Week 4 |
| PSO11 | 9 | 9 | 8 | 138 | 45 | 27 |
| PSO13 | 8 | 8 | 8 | 186 | 26 | 18 |
| PSO23 | 7 | 8 | 9 | 24 | 38 | 6 |
| PSO18 | 8 | 9 | 8 | 760 | 1052 | 1149 |
| PSO4 | 9 | 9 | 9 | 5 | 7 | 6 |
| PSO15 | 8 | 7 | 7 | 59 | 37 | 57 |
| PSO7 | 9 | 7 | 9 | 261 | 44 | 609 |
| PSO19 | 7 | 9 | 9 | 122 | 51 | 15 |
| PSO24 | 8 | 7 | 8 | 133 | 82 | 110 |
| PSO3 | 8 | 8 | 8 | 111 | 116 | 301 |
| PSO14 | 9 | 8 | 7 | 20 | 40 | 41 |
| PSO28 | 8 | 8 | 9 | 213 | 137 | 523 |
| PSO9 | 9 | 6 | 7 | 486 | 542 | 256 |
| PSO22 | 7 | 8 | 7 | 33 | 41 | 164 |
| PSO1 | 9 | 9 | 9 | 642 | 907 | 539 |
| PSO27 | 7 | 9 | 9 | 55 | 30 | 375 |
| PSO20 | 8 | 7 | 9 | 223 | 797 | 149 |
| PSO29 | 10 | 9 | 9 | 18 | 210 | 25 |
| PSO30 | 9 | 9 | 10 | 30 | 7 | 8 |
| PSO16 | 9 | 9 | 9 | 49 | 37 | 26 |
| PSO5 | 10 | 8 | 10 | 48 | 97 | 58 |
| PSO8 | 9 | 10 | 9 | 75 | 12 | 22 |
| PSO2 | 8 | 9 | 7 | 41 | 4 | 32 |
| PSO26 | 9 | 9 | 9 | 86 | 100 | 41 |
| PSO6 | 9 | 9 | 8 | 195 | 74 | 368 |
| PSO10 | 9 | 9 | 9 | 74 | 44 | 35 |
| PSO12 | 8 | 9 | 9 | 143 | 171 | 116 |
| PSO31 | 9 | 9 | 9 | 46 | 91 | 23 |
| PSO21 | 7 | 8 | 9 | 60 | 63 | 12 |
| PSO17 | 9 | 9 | 8 | 5 | 357 | 272 |
| PSO25 | 9 | 9 | 9 | 366 | 106 | 51 |
| HV 1 | 10 | | | 278 | | |
| HV 2 | 10 | | | 684 | | |
| HV 3 | 10 | | | 369 | | |
| HV 4 | 10 | | | 425 | | |
| HV 5 | 10 | | | 232 | | |
| HV 6 | 10 | | | 348 | | |
| HV 7 | 10 | | | 526 | | |
| HV 8 | 10 | | | 355 | | |

TABLE SUPPLEMENTARY S2

| | | | Fold increase | Fold increase | |
|---|---|---|---|---|---|
| | | | in psoriasis | in psoriasis | |
| Gene | Name | Function | skin | hair follicles | P value |
| IL8 | interleukin 8 | inflammatory response/ | 13.9 | 29.5 | <0.001 |
| | | chemotaxis | | | |
| DEFB4 | defensin, beta 4 | immune response/defense | 5.7 | 20.3 | <0.001 |
| | | response | | | |
| S100A7 | S100 calcium binding | epidermis development/ | 296* | 11.1 | <0.001 |
| | protein A7 | angiogenesis | | | |
| S100A9 | S100 calcium binding | inflammatory response | 3.1 | 6.3 | <0.001 |
| | protein A9 (calgranulin B) | | | | |
| S100A12 | S100 calcium binding | inflammatory response | 33.6 | 5.0 | 0.003 |
| | protein A12 (calgranulin | | | | |
| | C) | | | | |
| IL1B | interleukin 1, beta | inflammatory response/apoptosis | 9.1* | 2.6 | 0.07 |
| TNFa | tumor necrosis factor | immune response/anti-apoptosis | 5.5* | 2.1 | 0.03 |
| | alpha | | | | |
| IFIT3 | Interferon-induced protein | Unknown | 3.1 | 2.1 | 0.03 |
| | with tetratricopeptide | | | | |
| | repeats 3 | | | | |
| LCN2 | lipocalin 2 (oncogene | modulator of inflammation | 9.9 | 2.0 | 0.001 |
| | 24p3) | | | | |
| MMP9 | matrix metallopeptidase 9 | extracellular matrix organization | 4.2 | 1.9 | 0.3 |
| | (gelatinase B, 92 kDa | and biogenesis | | | |
| | gelatinase, 92 kDa type IV | | | | |
| | collagenase) | | | | |
| TCN1 | transcobalamin I (vitamin | cobalamin transport | 28.1 | 1.9 | 0.2 |
| | B12 binding protein, R | | | | |
| | binder family) | | | | |
| IFI27 | interferon, alpha-inducible | ND | 3.5 | 1.9 | 0.002 |
| | protein 27 | | | | |
| PBEF1 | pre-B-cell colony | positive regulation of cell | 3.4 | 1.8 | 0.004 |
| | enhancing factor 1 | proliferation/cell-cell signalling | | | |
| HPSE | heparanase | proteoglycan metabolic process | 4.9 | 1.8 | 0.01 |
| PPARD | peroxisome proliferator- | fatty acid catabolic and epidermis | 5.5* | 1.8 | 0.01 |
| | activated receptor delta | proliferation | | | |
| IL1F9 | interleukin 1 family, | cell-cell signalling | 11.2 | 1.7 | |
| | member 9 | | | | |
| SERPINB3 | serpin peptidase inhibitor, | serine-type endopeptidase | 8.9 | 1.7 | |
| | clade B (ovalbumin), | inhibitor activity | | | |
| | member 3 | | | | |
| KLK13 | kallikrein 13 | proteolysis | 5.3 | 1.7 | |
| SOD2 | superoxide dismutase 2, | response to oxidative stress | 3.2 | 1.6 | |
| | mitochondrial | | | | |
| SERPINB1 | serpin peptidase inhibitor, | serine-type endopeptidase | 5.8 | 1.4 | |
| | clade B (ovalbumin), | inhibitor activity | | | |
| | member 1 | | | | |
| ECGF1 | endothelial cell growth | angiogenesis/pyrimidine | 6.5 | 1.2 | |
| | factor 1 (platelet-derived) | nucleotide metabolic process | | | |
| LTBP1 | Latent transforming | TGFb signalling | 3.4 | 1.2 | |
| | growth factor beta binding | | | | |
| | protein 1 | | | | |
| SPRR1A | small proline-rich protein | keratinocyte differentiation | 3.1 | 1.2 | |
| | 1A | | | | |
| MX1 | myxovirus (influenza virus) | defense response/induction of | 4.3 | 1.2 | |
| | resistance 1, interferon- | apoptosis | | | |
| | inducible protein p78 | | | | |
| | (mouse) | | | | |
| STAT1 | signal transducer and | regulation of progression through | 3.7 | 1.1 | |
| | activator of transcription 1, | cell cycle/transcription from RNA | | | |
| | 91 kDa | polymerase II promoter | | | |
| KLK6 | kallikrein 6 (neurosin,) | collagen catabolic process | 4.5 | 1.1 | |
| TGM1 | transglutaminase 1 | keratinocyte differentiation | 3.4 | 1.1 | |
| DSG3 | desmoglein 3 (pemphigus | cell adhesion | 3 | 1.0 | |
| | vulgaris antigen) | | | | |
| DSC2 | desmocollin 2 | cell adhesion | 5.1 | 0.9 | |
| SLC6A14 | solute carrier family 6 | amino acid metabolic process | 7.4 | 0.9 | |
| | (amino acid transporter), | | | | |
| | member 14 | | | | |
| AKR1B10 | aldo-keto reductase family | steroid metabolic process | 6.1 | 0.9 | |
| | 1, member B10 (aldose | | | | |
| | reductase) | | | | |
| IL1RN | interleukin 1 receptor | immune response | 3 | 0.8 | |
| | antagonist | | | | |
| GJB2 | gap junction protein, beta | Cell communication | 50.5 | 0.8 | |
| | 2, 26 kDa (connexin 26) | | | | |
| KRT16 | keratin 16 (focal non- | epidermis development | 3.7 | 0.8 | |
| | epidermolytic | | | | |
| | palmoplantar | | | | |
| | keratoderma) | | | | |
| FABP5 | fatty acid binding protein 5 | epidermis development | 5.3 | 0.7 | |
| | (psoriasis-associated) | | | | |
| IL1F5 | interleukin 1 family, | inflammatory response | 3.9 | 0.6 | |
| | member 5 (delta) | | | | |
| WNT5A | wingless-type MMTV | Wnt receptor signalling pathway | 3.4 | 0.5 | |
| | integration site family, | | | | |
| | member 5A | | | | |
| KRT6E | keratin 6E | cytoskeleton organization and | 6.8 | 0.3 | |
| | | biogenesis | | | |
| IFNG | interferon, gamma | immune response | 5.7* | undetected | |
| CCL20 | chemokine (C-C motif) | inflammatory response/ | 4.5 | undetected | |
| | ligand 20 | chemotaxis | | | |
| CHI3L2 | chitinase 3-like 2 | carbohydrate metabolic process | 3.6 | undetected | |
| SPRR2D | small proline-rich protein | epidermis development/ | 3.3 | undetected | |
| | 2D | keratinocyte differentiation | | | |
| RASGRP2 | RAS guanyl releasing | regulation of cell growth/Ras | 16.1 | undetected | |
| | protein 2 (calcium and | protein signal transduction | | | |
| | DAG-regulated) | | | | |
| CXCL10 | chemokine (C—X—C motif) | inflammatory response/ | 22* | undetected | |
| | ligand 10 | chemotaxis | | | |

What is claimed is:

1. A technique for conducting non-invasive skin inflammatory disease/disorders pharmaco-genomic studies, comprising the steps of:
collecting hair follicles non-invasively, and
analyzing gene expression profiling thereof.

2. The technique as defined by claim 1, wherein the disease studied is psoriasis.

3. The technique as defined by claim 1, wherein the hair is collected from the scalp.

4. A method to monitor efficacy of a pharmacological agent in preventing or treating inflammatory skin disease/disorders, comprising the steps of:
administering to a patient in need of such treatment a therapeutically effective amount of a pharmacological agent,
collect non-invasively hair follicles from such patient,
study/determine the gene expression thereof by analysis,
analyze skin affected patient(s') samples to controls' samples or to previous skin affected patient(s') samples without pharmacological agent.

5. Evaluation efficacy method of a pharmacological agent as defined by claim 4, wherein the pharmacological agent is selected from a small molecule drug or a biological agent.

6. The technique as defined by claim 4, wherein the disease is psoriasis.

7. The technique as defined by claim 4, wherein the hair is collected from the scalp.

8. A non-invasive diagnosis of inflammatory skin disease/disorders, comprising the steps of:
collect non-invasively hair follicles,
study/determine the gene expression thereof by analysis,
clusterize control samples to skin affected patient(s') samples (specific embodiment psoriatic samples) based discriminating genes.

9. The diagnosis as defined by claim 7, wherein the disease is psoriasis.

10. The diagnosis as defined by claim 7, wherein the hair is collected from the scalp.

11. The diagnosis as defined by claim 7, wherein the analyzing method is Real time PCR.

12. The diagnosis as defined by claim 7, wherein discriminating genes are selected in the following: Keratin 16 (KRT16); gap junction protein, beta 2, (connexin 26) (GJB2); chitinase 3-like 2 (CHI3L2); interleukin 8 (IL8); fatty acid binding protein 5 (FABP5); interleukin 1, beta (IL1B); signal transducer and activator of transcription (STAT1); heparanase (HPSE); solute carrier family 6 (amino acid transporter), member 14 (SLC6A14); transcobalamin I (vitamin B12 binding protein, R binder family) (TCN1); tumor necrosis factor (TNF); interleukin 1 family, member 5 (delta) (ID1F5); small proline-rich protein 2D (SPRR2D); kallikrein 13 (KLK13); chemokine (C-X-C motif) ligand 10 (CXCL10); desmoglein 3 (pemphigus vulgaris antigen) (DSG3); S100 calcium binding protein Al 2 (S100A12); interleukin 1 receptor antagonist (IL1RN); superoxide dismutase 2, mitochondrial (SOD2); keratin 6C; (KRT6E); interferon-induced protein with tetratricopeptide repeats 3 (IFIT3); desmocollin 2 (DSC2); endothelial cell growth factor 1 (platelet-derived) (ECGF1); RAS guanyl releasing protein 2 (calcium and DAG-regulated) (RASGRP2); wingless-type MMTV integration site family, member 5A (WNT5A); myxovirus (influenza virus) resistance 1, interferon-inducible protein p78 (mouse) (MX1); small proline-rich protein 1A (SPRR1A); defensin, beta 4 (DEFB4); S100 calcium binding protein A9 (S100A9); interleukin 1 family, member 9 (IL1F9); kallikrein 6 (neurosin, zyme) (KLK6); matrix metallopeptidase 9 (MMP9); serpin peptidase inhibitor, clade B (ovalbumin), member 3 (SERPINB3); interferon, gamma (IFNG); lipocalin 2 (oncogene 24p3) (LCN2); interferon, alpha-inducible protein 27 (IFI27); peroxisome proliferator-activated receptor delta (PPARD); serpin peptidase inhibitor, clade B (ovalbumin), member 1 (SERPINB1); latent transforming growth factor beta binding protein 1 (LTBP1); pre-B-cell colony enhancing factor 1 (PBEF1); transglutaminase 1 (K polypeptide epidermal type I, protein-glutamine-gamma-glutamyltransferase) (TGM1); chemokine (C-C motif) ligand 20 (CCL20); aldo-keto reductase family 1, member B10 (aldose reductase) (AKR1B10); S100 calcium binding protein A7 (S100A7).

13. The diagnosis as defined by claim 8, wherein discriminating genes are selected in the following: interleukin 8 (IL8); beta 4defensin (DEFB4); S100 calcium binding protein A7 (S100A7); S100 calcium binding protein A9 (calgranulin B) (S100A9); S100 calcium binding protein A12 (S100A12); interleukin 1b (IL-1b); lipocalin 2 (oncogene 24p3) (LCN2); transcobalamin I (vitamin B12 binding protein, R binder family) (TCN1); Interferon alpha-inducible protein 27 (IFI27); Peroxisome proliferator-activated receptor- [symbol] PPAR-δ); serpin peptidase inhibitor, clade B (ovalbumin), member 3(SERPIN B3).

14. A method to monitor skin or scalp inflammation in a skin affected patient, comprising the steps of:
collect non-invasively patient's hair follicles,
study/determine the gene expression by analysis,
analyze inflammation gene (s) from said inflammatory skin affected patient(s') samples to controls' samples.

15. A method to monitor skin or scalp inflammation in a psoriatic patient, comprising the steps of:
collect non-invasively patient's hair follicles,
study/determine the gene expression by analysis,
analyze inflammation gene (s) from said inflammatory skin affected patient(s') samples to controls' samples or to previous patient psoriatic samples.

16. A predictive model of inflammatory skin affected patient(s') determination, comprising monitoring the modulation in expression of at least 1 discriminating genes or markers selected from the inflammatory markers.

17. The predictive model as defined by claim 16, wherein inflammatory skin affected patient(s') is psoriatic patient.

18. Discriminating genes or markers included in the model as defined by claim 17, selected from the following: interleukin 8 (IL8); beta 4defensin (DEFB4); S100 calcium binding protein A7 (S100A7); S100 calcium binding protein A9 (calgranulin B) (S100A9); S100 calcium binding protein A12 (S100A12); interleukin 1b (IL-1b); lipocalin 2 (oncogene 24p3) (LCN2); transcobalamin I (vitamin B12 binding protein, R binder family) (TCN1); Interferon alpha-inducible protein 27 (IFI27); Peroxisome proliferator-activated receptor- [symbol] PPAR-δ); serpin peptidase inhibitor, clade B (ovalbumin), member 3 (SERPIN B3).

19. Psoriatic scalp lesions biomarkers and/or gene expression products as biomarkers selected from among the following: interleukin 8 (IL8); beta 4defensin (DEFB4); S100 calcium binding protein A7 (S100A7); S100 calcium binding protein A9 (calgranulin B) (S100A9); S100 calcium binding protein A12 (S100A12); interleukin 1b (IL-1b); lipocalin 2 (oncogene 24p3) (LCN2); transcobalamin I (vitamin B12 binding protein, R binder family) (TCN1); Interferon alpha-inducible protein 27 (IFI27); Peroxisome proliferator-activated receptor- [symbol] PPAR-δ); serpin peptidase inhibitor, clade B (ovalbumin), member 3 (SERPIN B3).
